(19) United States
(12) Patent Application Publication
(10) Pub. No.: US 2014/0011985 A1
(43) Pub. Date: Jan. 9, 2014

(54) Targeted Cancer Therapy Conjugates Using Porphyrins and Porphyrin-like Molecules and Various Cytotoxic Agents

(71) Applicants: Maria Zannes, Albuqerque, NM (US); John J. Cousins, Albuquerque, NM (US); Timothy Peter Zannes, Albuquerque, NM (US); Gordon Bennett, Albuquerque, NM (US)

(72) Inventors: Maria Zannes, Albuqerque, NM (US); John J. Cousins, Albuquerque, NM (US); Timothy Peter Zannes, Albuquerque, NM (US); Gordon Bennett, Albuquerque, NM (US)

(21) Appl. No.: 13/544,137

(22) Filed: Jul. 9, 2012

(51) Int. Cl. C07D 487/22; C07F 5/00; B82Y 5/00

(52) U.S. Cl. 534/15; 540/145; 977/915

(57) ABSTRACT

Disclosed are targeted in-vivo cancer therapeutics based on the creating conjugate combinations of a porphyrin or porphyrin-like molecule or compound used as a delivery vehicle or vector, coupled with a cytotoxic agent that is released upon binding to a tumor site. The families of porphyrin or porphyrin-like molecules or compounds have a high affinity to bind with cancer cells and are organic structures and are not toxic or rejected by the body. The structure includes a binding site for metals and nano-particles. Benefits of the current invention include: accurate, targeted in-vivo radio and therapeutic agents delivered directly to tumor sites. A benefit of targeted therapy is sparing healthy cells and tissue, thus keeping the host patient healthier which enhances the therapeutic effects of reducing and destroying the cancer tumors. An additional benefit is keeping the patient from discomfort of sickness and the side effects of traditional radio and chemo-therapies.

### BACKGROUND OF THE INVENTION

1. Field of the Invention

The present invention is a system and methods for targeted cancer drug therapies using porphyrins and porphyrin-like compounds in conjugation with certain cytotoxins and therapeutic compounds for the purpose of targeted therapy of human cancers.

2. Description of the Prior Art

U.S. Pat. No. 4,649,151 relates to diagnosis and treatment with photosensitizing drugs by which tumors are detected and treated by irradiating the tumors with light after the drug accumulates in the tumor.

This patent does not teach the use of porphyrins as a transport vector for use with radioisotopes, heavy metals or nano-particles as the toxic agent in the diagnosis nor treatment of cancer.

U.S. Pat. No. 4,889,120 describes the application of the methods to create a substitution of naturally occurring metal moieties within porphyrins and porphyrin-like molecules with selected radioactive metals and metallic rare earths.

This patent does not teach or describe a method or system for using these compounds for creating in vivo cancer therapy drugs.

U.S. Pat. No. 5,238,940 describes irradiation of cancers cells after identification and binding by the porphyrin compound. Conjugates of porphyrin-type photosensitizers and hydrophilic polymers used as active ingredients in the compositions are used to effect the destruction or impairment of suitable target moieties such as cancer cells and viruses.

This patent does not teach the use of porphyrins as a transport vector or the use of radioisotopes, heavy metals or nano-particles as the toxic agent in the diagnosis nor treatment of cancer.

U.S. Pat. No. 5,258,453 describes a drug delivery system for the simultaneous delivery of drugs activatable by enzymes and light. Compositions containing both an anticancer drug and a photoactivatable drug are attached to copolymeric carriers and administered to the patient. Then a light source of the appropriate wavelength and energy is to activate the photoactivatable drug.

This patent does not teach the use of porphyrins as a transport vector or the use of radioisotopes, heavy metals or nano-particles as the toxic agent in the diagnosis nor treatment of cancer.

U.S. Pat. No. 5,541,297 describes the methods for producing and use of a drug or modified toxin and a protein which reacts with a substance associated with a targeted cell or pathogen. The targeted substance internalizes the conjugate into the cell cytoplasm, and then kills the cell.

This patent does not teach the use of porphyrins as a transport vector or the use of radioisotopes, heavy metals or nano-particles as the toxic agent in the diagnosis nor treatment of cancer.

U.S. Pat. No. 5,573,909 relates to methods for labeling or detecting one or more target materials using surface coated fluorescent microparticles with unique characteristics. The unique microparticles are combined with a sample thought to contain the target material(s), so that the microparticles label the target materials. The sample is then optionally illuminated, resulting in fluorescence of the microparticles that is used to detect one or more target materials.

This patent does not teach the use of porphyrins as a transport vector or the use of radioisotopes, heavy metals or nano-particles as the toxic agent in the diagnosis nor treatment of cancer.

U.S. Pat. No. 5,601,825 describes the methods for producing and use of a conjugate including use of a drug or modified toxin and a protein. The targeted substance internalizes the conjugate into the cell cytoplasm, and the kills the cell.

This patent does not teach the use of porphyrins as a transport vector or the use of radioisotopes, heavy metals or nano-particles as the toxic agent.

U.S. Pat. No. 6,693,093 describes the compositions and methods for drug formulations, storage and delivery methods useful for photodynamic therapy (PDT) utilizing photosensitizer drugs and one or more block copolymers as carriers for drugs in the treatment of diseases.

This patent does not teach the use of porphyrins as a transport vector or the use of radioisotopes, heavy metals or nano-particles as the toxic agent in the diagnosis nor treatment of cancer.

U.S. Pat. No. 7,026,347 describes the making and using of porphyrin-polyamine conjugate compounds that have anticancer and antitumor effects. The porphyrin moiety selectively localizes in tumors, while the polyamine moiety serves as a cytotoxic agent.

This patent does not teach the use of cytotoxins such as radioisotopes, rare earths or nano-particles as the therapeutic agent of the conjugate for the purpose of targeting and delivering a drug or toxic agent as a medical therapy for multiple cancers.

U.S. Pat. No. 7,344,710 describes the use of anaerobic bacteria strain of C. novyi devoid of its lethal toxin (C. novyi-NT), a strategy called combination bacteriolytic therapy (COBALT), intravenously injected within the avascular regions of tumors to destroy surrounding viable tumor cells.

This patent does not teach the use of porphyrins as a transport vector or the use of radioisotopes, heavy metals or nano-particles as the toxic agent.

U.S. Pat. No. 7,682,603 is a patent describing the use of compositions comprising polysomes as a targeting moiety with surface attached visible or nearly infrared agents for the treatment of disease and imaging methodology.

This patent does not teach or describe the use of porphyrins for the purpose of targeting and delivering a drug or toxic agent as a medical therapy for cancer treatment.

U.S. Pat. No. 7,820,143 is a patent that describes an invention for a method of treatment for disease by photodynamic therapy with a light after injecting a compound possibly including a porphyrin and treating the affected area with a light source that excites the compound.

This patent does not teach the use of cytotoxins such as radioisotopes, rare earths or nano-particles as the therapeutic agent of the conjugate for the purpose of targeting and delivering a drug or toxic agent as a medical therapy for multiple cancers.

U.S. Pat. No. 8,148,335 is a patent that describes the use of nano-particles for drug delivery for treatment.

This patent does not teach the use of porphyrins for the purpose of targeting and delivering a drug or other toxic agent as a medical therapy for cancer therapies.

U.S. Pat. No. 8,168,586 describes methods and compositions for assessing, treating and preventing diseases using cancer associated targets.

This patent does not teach or describe the use of porphyrins for the purpose of targeting and delivering a drug or toxic agent as a medical therapy for cancer treatment.

U.S. Pat. No. 8,180,436 relates to apparatus and devices for florescent based imaging and ablation of medical targets.

This patent does not teach the use of porphyrins for the purpose of targeting and delivering a drug or toxic agent as a medical therapy for cancer treatment.

U.S. Pat. No. 8,188,239 describes the composition of targetable constructs that are multivariate carriers of bi-specific antibodies for the treatment of cancer and autoimmune diseases.

The patent does not involve the use of porphyrins and porphyrin-like compounds as a target device to carry cytotoxins such as radioisotopes, rare earths or nano-particles as the therapeutic agent of the conjugate for the purpose of targeting and delivering a drug or toxic agent as a medical therapy for multiple cancers.

U.S. Pat. No. 8,198,246 discloses a pharmaceutical composition of liposome nano-particles for lodging in a target tissue cell in situ of an animal subject as a targeting delivery system for physical cancer therapy.

This patent does not teach or describe the use of porphyrins for the purpose of targeting and delivering a drug or toxic agent as a medical therapy for cancer treatment.

U.S. Patent Applications Number 2011/0243914 and Number 2010/0329992 describe the invention of a new compound for cancer therapy or diagnosis and more specifically to the use of a non-toxic B subunit of Shiga toxin mutant as a vector for diagnostic products or drugs in over-expressing Gb.sub.3 receptor cells, including use of porphyrins for the identification of receptor sites.

The patent does not involve the use of porphyrins and porphyrin-like compounds as a target device to carry cytotoxins such as radioisotopes, rare earths or nano-particles as the therapeutic agent of the conjugate for the purpose of targeting and delivering a drug or toxic agent as a medical therapy for multiple cancers.

### SUMMARY OF THE INVENTION

The present invention is a system and methods for targeted in-vivo cancer therapeutics based on the creating conjugate combinations of a porphyrin or porphyrin-like molecule or compound used as a delivery vehicle or vector, and a cytotoxic agent that is released upon binding to a tumor site. The families of porphyrin or porphyrin-like molecules or compounds have a high affinity to bind with cancer cells and are organic structures similar to the hema in hemoglobin and as such are not toxic or rejected by the body in this in-vivo application. The ring structure of the porphyrin molecule also creates a binding site for metals such as radioactive metals and metallic rare earths, and nano-particles. After attachment to the tumor, the metals can be heated with tuned lasers to release toxins that selectively destroy the tumor.

Examples of some of the radioactive metals and metallic rare earths, but not limited to these stated, which can be used are: yttrium 90, gadolinium 153, cobalt 57, cobalt 58, cobalt 60, and technetium-99m.

Benefits of the current invention include: accurate, targeted in-vivo radio and chemo-therapy agents delivered directly to tumor sites. This system will provide benefits to cancer patients by providing targeted therapy and sparing healthy cells and tissue during the treatment process, thus keeping the host patient healthier. Targeted delivery of cancer drugs and cytotoxins to the tumor site will enhance the beneficial outcome of cancer treatment and therapy.

An additional benefit is keeping the patient from discomfort of sickness and the side effects of traditional radio and chemo-therapies.

It is therefore a primary object of the present invention to provide highly effective and accurate, targeted cancer therapies delivered in the body of the diseased patient.

It is another object of the present invention to identify and create in-vivo transport mechanisms and delivery vehicles that have high specificity and sensitivity to attach to tumor sites.

It is another object of the present invention to attach radioactive metals and metallic rare earths to the delivery mechanisms to create cancer therapy compounds.

It is another object of the present invention to implement these cancer therapy compounds in therapy regiments to reduce and eliminate tumors.

It is another object of the present invention to create an embodiment of the invention for use with gold nano-particles that are heated via a resonance tuned laser targeted on the tumor site after the nano-particle/delivery agent have been bound to the tumor site.

It is another object of the present invention to attach nano-particles to the delivery mechanisms to create cancer therapy compounds.

It is another object of the present invention to create an embodiment of the invention for use with toxins that are released after the compound has been delivered and bound to the tumor site.

It is another object of the present invention to create processes to introduce these hybrid therapy compounds into the patient such as injectables into the bloodstream; inhalers using aerosols and nebulizers, and It is another object of the present invention to create processes for tracking the location of the therapy compounds within the patient's body and identifying the locus of binding at tumor sites.

It is another object of the present invention to decrease the number of therapy sessions and time of therapy.

It is another object of the present invention to limit the exposure of healthy tissue and cells to toxins and radioactive procedures meant to destroy cancer tumors and cells.

It is another object of the present invention to increase ability to calibrate targeted dosages in order to minimize exposure to toxic elements in order to reduce side effects.

It is another object of the present invention to create efficiencies and cost savings given that the average therapy time and sessions will be significantly reduced.

These and other objects of the present invention will become apparent to those skilled in this art upon reading the accompanying description, drawings, and claims set forth herein.

### BRIEF DESCRIPTION OF THE DRAWINGS

FIG. 1 is a systems diagram detailing the best mode of the various sub systems of the present invention. The figure shows the porphyrin and porphyrin-like compounds as the transport vector and the cytotoxin which are conjugated and then introduced into the patient where the therapeutic conjugate binds to the tumor and performs its cytotoxic effect.

FIG. 2 illustrates the process of the conjugate locating and binding to the tumor site and killing tumor cells.

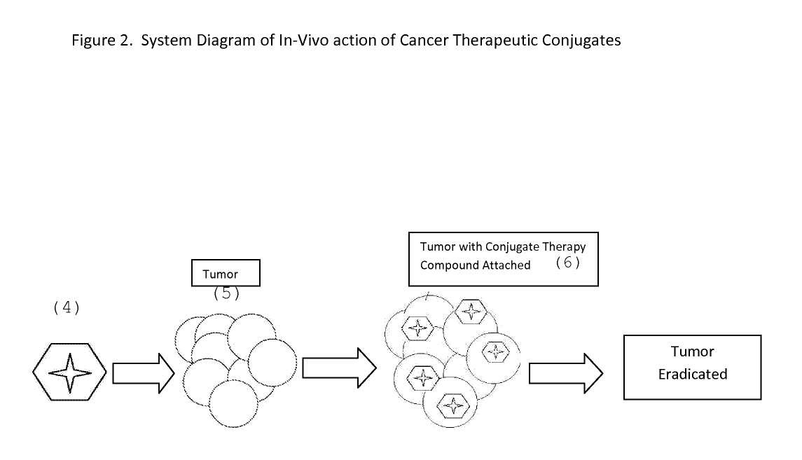

### DETAILED DESCRIPTION OF THE INVENTION

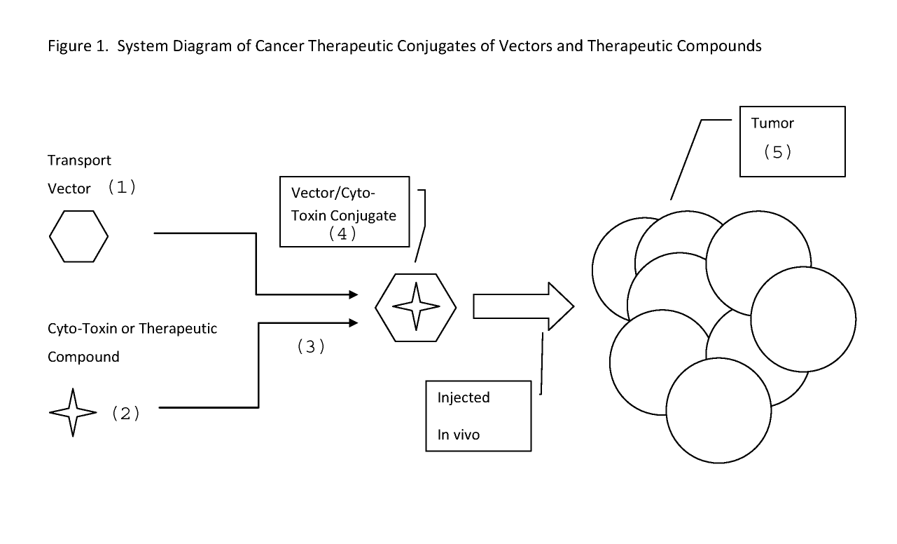

The best mode of the invention as shown in FIG. 1 is a system and methods for the application of porphyrins and porphyrin-like molecules (1) wherein certain molecules or compounds (2) are substituted for the naturally occurring metal moiety (3), such as iron in the hema of hemoglobin, of such porphyrins and porphyrin-like molecules and compounds for the purpose of therapy of human cancers (5).

The present invention is a system and methods for in-vivo cancer therapeutics based on the combination of a porphyrin or porphyrin-like molecule or compound used as a delivery vehicle or vector (1), coupled with a toxic agent (2) that is released upon binding to a tumor site (6). The families of porphyrin or porphyrin-like molecules or compounds have a high affinity to bind with cancer cells and are organic structures similar to the hema in hemoglobin and as such are not toxic or rejected by the body in this in-vivo application. The ring structure also creates a binding site for metals (3) such as radioactive metals and metallic rare earths; and nano-particles that can act as tumor destroyers by being heated with tuned lasers or by releasing toxins after attaching to tumors.

### Delivery Vectors

Certain porphyrins and porphyrin-like molecules and compounds (1) such as, but not limited to: etioporphyrins, mesoporphyrins, coproporphyrins, uroporphyrins, protoporphyrins, dicarboxylic acid containing porphyrins, substituted porphyrins such as tetraphenylporphyrin sulfonate and protoporphyrin containing molecules such as hematoporphyrins, chlorophylls, cytochromes and combinations thereof, can be selected for their abilities to be taken up and selectively and preferentially retained by pre-cancerous and cancerous human tissue (6).

### Cytotoxins

Some of the examples of the radioactive metals and metallic rare earths that can be used as cytotoxic therapeutic agents (2) claimed here, but not limited to these examples, are:
yttrium-90 substituted into the selected porphyrin or porphyrin-like molecule or compound for the purpose in vivo treatment of human cancers and precancerous conditions.
cobalt-57 substituted into the selected porphyrin or porphyrin-like molecule or compound for the purpose of in vivo application of the porphyrin/cobalt-57 complex as a radio-therapeutic agent as gamma radiation is cytotoxic.
cobalt-60 substituted into the selected porphyrin or porphyrin-like molecule or compound for the purpose of in vivo application of the porphyrin/cobalt-57 complex as a radio-therapeutic agent as gamma radiation is cytotoxic.

Nano-particles (2) can be used in several modalities. An example is Gold nano-particles can be used as the cytotoxin in the conjugate that can be exited with a tuned laser at the resonant frequencies and the resultant heat used to kill cancer cells.

Nano-particles (2) can also be used to carry cytotoxins that are then released after the vector is bound to the tumor site. Release of the cytotoxin can be induced by laser excitation or other device.

### Benefits

Benefits of the current invention include: accurate, targeted in-vivo radio and chemo-therapy agents delivered directly to tumor sites (6). This system will provide benefits to cancer patients by providing targeted therapy and sparing healthy cells and tissue, thus keeping the host patient healthier which will aid in enhancing the therapeutic effects of the toxic agents destroying the cancer tumors. An additional benefit is keeping the patient from discomfort of sickness and the side effects of traditional radio and chemo-therapies.

What is claimed is:

1. A system for creating targeted cancer therapeutic conjugate compounds utilizing the non-toxic, preferential binding to cancer cells and binding sites of porphyrins and porphyrin-like molecules, to bind and transport cytotoxins in vivo to tumor sites for targeted cell necrosis.

2. A system according to claim 1 using porphyrins and porphyrin-like molecules as the binding and transport mechanism.

3. A system according to claim 1 using radio isotopes as the cytotoxin.

4. A system according to claim 1 using rare earth metals as the cytotoxin.

5. A system according to claim 1 using nano-particles as the cytotoxin.

6. A system according to claim 1 to create the conjugate from the constituent parts.

7. A system according to claim 1 to create a suspension of the conjugate therapeutic compound for introduction into the patient.

8. A system according to claim 1 to introduce the conjugate therapeutic compound into the patient via injection.

9. A system according to claim 1 to introduce the conjugate therapeutic compound into the patient via inhaler or nebulizer.

10. A system according to claim 1 to locate and identify the conjugate therapeutic compound once it is bound and attached to the tumor site.

11. A Method for creating targeted cancer therapeutic conjugate compounds utilizing the non-toxic, preferential binding to cancer cells and binding sites of porphyrins and porphyrin-like molecules, to bind and transport cytotoxins in vivo to tumor sites for targeted cell necrosis.

12. A method according to claim 11 using porphyrins and porphyrin-like molecules as the binding and transport mechanism.

13. A method according to claim 11 using radio isotopes as the cytotoxin.

14. A method according to claim 11 using rare earth metals as the cytotoxin.

15. A method according to claim 11 using nano-particles as the cytotoxin.

16. A method according to claim 11 to create the conjugate from the constituent parts.

17. A method according to claim 11 to create a suspension of the conjugate therapeutic compound for introduction into the patient.

18. A method according to claim 11 to introduce the conjugate therapeutic compound into the patient via injection.

19. A method according to claim 11 to introduce the conjugate therapeutic compound into the patient via inhaler or nebulizer.

20. A method according to claim 11 to locate and identify the conjugate therapeutic compound once it is bound and attached to the tumor site.
